(19) United States
(12) Patent Application Publication
(10) Pub. No.: US 2009/0110656 A1
(43) Pub. Date: Apr. 30, 2009

(54) Skin cooling composition

(76) Inventors: Sarah A. Lemke, Appleton, WI (US); Scott W. Wenzel, Neenah, WI (US); Tammy J. Balzar, Oshkosh, WI (US)

Correspondence Address: KIMBERLY-CLARK WORLDWIDE, INC.;Catherine E. Wolf, 401 NORTH LAKE STREET, NEENAH, WI, 54956, US

(21) Appl. No.: 11/981,344

(22) Filed: Oct. 31, 2007

(51) Int. Cl. A61K 31/722; A61P 43/00

(52) U.S. Cl. 424/78.02

(57) ABSTRACT

A cooling composition for application to the skin of a mammal, such as a human. The base of the composition is an interpenetrating polymer network, which may contain a thermo-responsive polymer. Having a Critical Temperature above an average core body temperature, the material may undergo a phase change as the creature becomes too warm, such as when a person experiences a hot flash, a fever, exposure to very warm environments, physical exertion, and the like. The phase change results in a cooling and/or cooling sensation of the skin. Additives may be included in the cooling composition such as evaporative cooling components, neurosensory cooling components, and additional phase change components.

### BACKGROUND OF THE INVENTION

The invention relates to a skin cooling composition, and in particular to a skin cooling composition that provides short and long term skin cooling.

There are a variety of products that are applied to the skin. It would be desirable under many circumstances if such products provided a cooling feeling to the skin when the products were applied to the skin. Existing products typically provide skin cooling by combining skin cooling agents with other substances. However, many existing products fail to provide satisfactorily strong and long lasting skin cooling, and may not be reused as desired.

There are several different ways to impart a cooling sensation to the skin, including the use of neurosensory components, physical agents such as phase change materials, and evaporative components. One example of a neurosensory component is menthol. The cooling sensation from menthol is not due to latent heat of evaporation, but appears to be the result of direct stimulus on the cold receptors at the nerve endings. One example of a phase change material is wax. Wax may provide cooling that eventually dissipates because it can no longer effectively phase change, or is spread too thin so that it is no longer sensed by the user. Finally, one example of an evaporative cooling component is alcohol.

Again, once applied, alcohol disappears by the process of evaporation.

For a person that experiences hot flashes or fluctuations in body temperature, relief may be obtained after a first application of the existing cooling compounds. However, reactivation may not be possible, even if the components are combined into a single compound. Thus, there is a need for skin cooling compositions that can be reactivated to provide at least intermittent cooling as the body changes temperature due to hot flashes or the like. There is a further need for a skin cooling composition that may be reused.

While particular aspects and/or individual features of the present invention have been illustrated and described, it would be obvious to those skilled in the art that various other changes and modifications can be made without departing from the spirit and scope of the invention. Further, it should be apparent that all combinations of such aspects and features are possible and can result in preferred executions of the invention.

### SUMMARY OF THE INVENTION

The compositions of the present invention are used to cool the skin of a mammal. One aspect of the present invention provides a temperature change composition having a thermo-responsive polymer. This thermo-responsive polymer is an interpenetrating polymer network that is in a liquid state at about Room Temperature, and a solid state at a temperature greater than about Core Body Temperature. The composition includes at least one additional cooling component.

Another aspect of the present invention provides a temperature change composition having a thermo-responsive polymer. This thermo-responsive polymer is an interpenetrating polymer network that is in a solid state at Room Temperature. The composition includes at least one additional cooling component.

Yet another aspect of the present invention provides a temperature change composition having a thermo-responsive polymer that includes a chitosan based hydrogel. The composition includes at least one additional cooling component, selected from the following: a phase change component, a first neurosensory cooling component, and an evaporative cooling component.

Additional features and advantages will be apparent from the detailed description which follows, taken into conjunction with the accompanying drawings, which together illustrate, by way of example, features of the invention.

### BRIEF DESCRIPTION OF THE DRAWINGS

A full and enabling disclosure of the present invention, including the best mode thereof, directed to one of ordinary skill in the art, is set forth more particularly in the remainder of the specification, which makes reference to the appended figures in which:

FIG. 1 is a chart showing the various possible components of the cooling composition of the present invention, having as a base, an interpenetrating polymer network;

FIG. 2 is a chart showing one embodiment of the present invention, that includes an interpenetrating network, a neurosensory component, and an evaporative cooling component; and

FIG. 3 is another embodiment of the present invention that includes an interpenetrating network, a neurosensory component, two phase-change materials, and an evaporative cooling component.

### DETAILED DESCRIPTION OF THE INVENTION

It is to be understood by one of ordinary skill in the art that the present discussion is a description of exemplary aspects of the present invention only, and is not intended as limiting the broader aspects of the present invention.

The term “Room Temperature” as used in the context of the present invention is defined as a range of about 20 degrees Celsius (“C”) to about 23 C. (about 68 degrees Fahrenheit (“F”) to about 74 F).

The term “Body Temperature” is the temperature at the skin surface of a user. For humans, this is a range of about 34 C. (93.2 F)±3 C. (87.8 F.-98.6 F.). The average “Core Body Temperature” for a human is about 37 C. (98.6 F.).

The invention relates to a skin cooling composition that may provide continuous or intermittent skin cooling. Intermittent skin cooling can occur when the skin surface temperature crosses more than once, a certain temperature threshold at which point a phase change occurs in the skin cooling composition. The composition may be readily applied to the skin in liquid or solid form, and does not require solid-substrate carrier, though it may be combined with one if desired. In another embodiment, the composition may be readily mixed with other compositions that can be applied to the skin.

A phase change material is broadly defined as any substance that has the capability of absorbing or releasing thermal energy to reduce or eliminate heat flow at or within a temperature stabilizing range. At least one phase change component of the present invention is a thermo-responsive polymer. In the context of the present invention, a thermo-responsive polymer is defined as a polymer that can, in a relatively short time period, change one or more of its properties at a particular temperature (hereinafter referred to as the “Critical Temperature”). In one embodiment of the present invention, a polymer solution below the Critical Temperature is prepared from a thermo-responsive polymer dissolved in a solvent. When heated to or beyond a Critical Temperature, the polymer changes to a different phase. For example, a phase change material may be in a liquid or solid state at Room Temperature. By way of example, if the phase change material temperature is raised to a temperature above the Core Body Temperature or Body Temperature, and that temperature is greater than or equal to the Critical Temperature, then a phase change will occur.

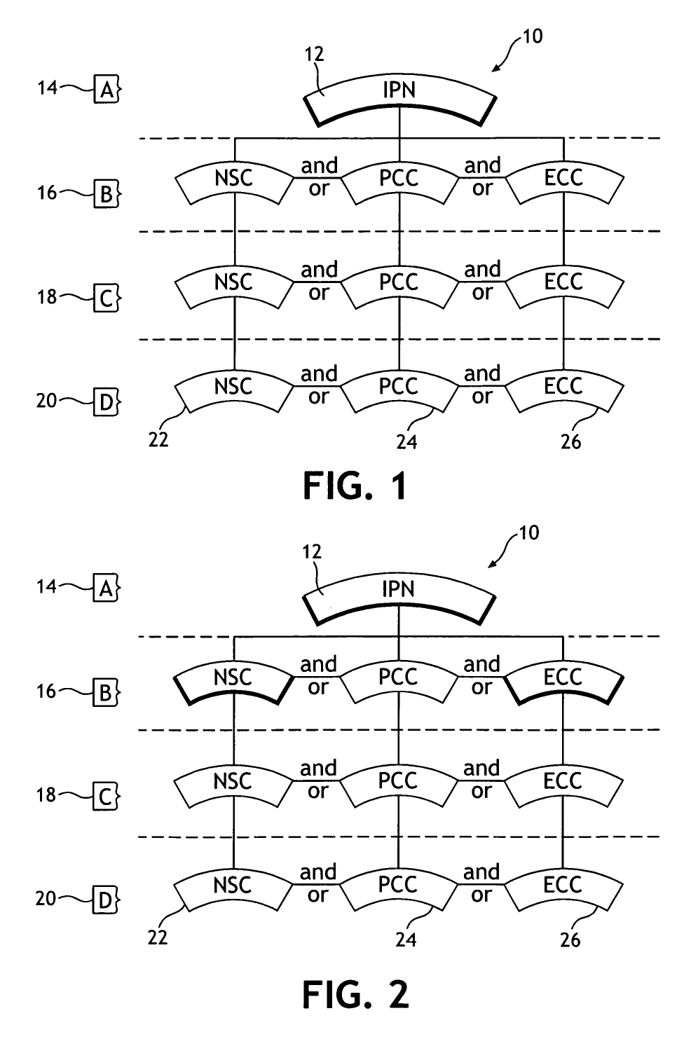

Referring to FIG. 1, in all embodiments of the present invention, the cooling composition 10 includes a particular kind of thermo-responsive polymer that is an interpenetrating polymer network 12 or “IPN 12.” IPN 12 operates as the base structure which will serve to provide the reactivatable (intermittent) cooling and possible reusable cooling aspects of the present invention. To this base structure, is one or more other components may be added, such as a neurosensory cooling component 22 “NSC 22,” a phase change component 24 “PCC 24,” and/or an evaporative cooling component 26 “ECC.”

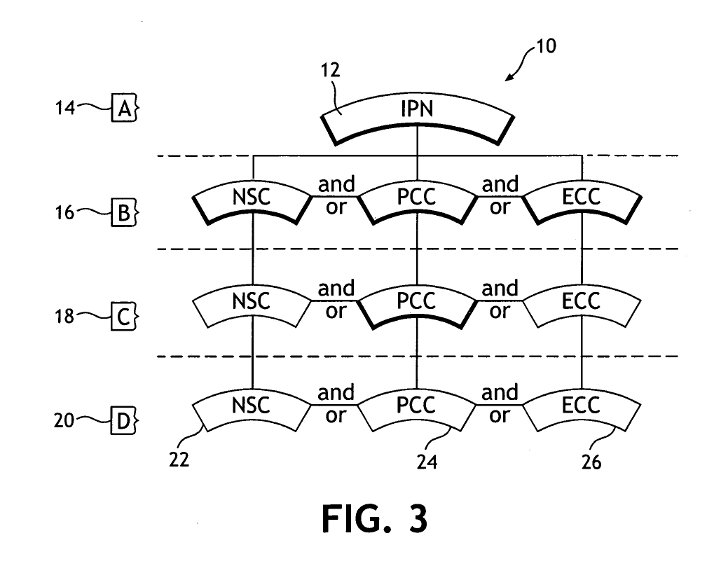

FIG. 1 demonstrates that there may be one or more neurosensory components 22, and/or one or more phase change components 24, and/or one or more evaporative cooling components 26 mixed into the base structure, IPN 12. In a non-limiting example, the IPN is referred to in FIG. 1 as level 14. At the next level 16, IPN 12 may include the following components: one NSC 22, one PCC 24, and/or one ECC 26. Likewise, at levels 18 and 20, it is possible to add one or more of the NSC 22, PCC 24, and/or ECC 26 components. As a specific example, FIG. 2 shows a cooling composition 10 having an IPN 12, and NSC 22, an ECC 26. In another example, FIG. 3 shows a cooling composition 10 having an IPN 12, two PCC's 24, an ECC 26 and an NSC 22. Details of these and other components or additives are described below.

Generally, an IPN 12 is a material containing two polymers, each in network form. IPN's 12 are distinguishable from blends, block copolymers, and graft copolymers in two ways: (1) an IPN 12 swells but does not dissolve in solvents, and (2) creep and flow are suppressed such that it will roughly hold the initial shape it has after application to the skin. The IPN 12 may change phase, such as between a solid and a liquid, multiple times without becoming “spent,” or somehow inoperative in this regard. An IPN may provide structure (e.g. form a skin patch), and may be applied to the skin as a solid or a liquid.

The temperature stabilizing range of IPN 12 may have a particular transition temperature or range of transition temperatures. The IPN 12 used in conjunction with various aspects of the present invention preferably will be capable of altering a flow of thermal energy during a time when the IPN 12 is absorbing or releasing heat, e.g. when IPN 12 undergoes a transition between two states such as liquid and solid states. This action is typically transient, meaning it will occur until a latent heat of the IPN 12 is absorbed or released during a heating or cooling process. Thermal energy may be stored or removed from the IPN 12, and the IPN 12 may typically be recharged by a heat source or a heat sink, depending on the actual IPN composition.

Suitable IPNs include but are not limited to (a) poly(acrylic acid) and a block copolymer of poly(ethylene oxide)/poly(propylene oxide); (b) a thermally reversible gel or thermally reversible gelling copolymer that is a random copolymer of a methacrylamide derivative and a hydrophilic comonomer, where the random copolymer is in the form of a plurality of linear chains having a plurality of molecular weights greater than or equal to a minimum gelling molecular weight cutoff; (c) reversible gels based on NiPAAM and copolymers that do not reverse upon dilution (they have a lower initial viscosity) (see U.S. Pat. No. 5,262,055 to Bae et al. incorporated by reference to the extent it does not conflict with the present invention); (d) poly methyl vinyl ether gel families; (e) a pH gelling cationic chitosan solution transformed into thermally sensitive, pH dependent, gel forming systems that are liquid at Room Temperature, made by addition of polyol salts (β-glycerophosphate, GP) at low polymer concentrations, e.g. 2 wt % (chitosan is the deaceylated derivative of chitin and is a biocompatible, pH dependent cationic polymer which is soluble in water up to pH 6.2); and (f) a pH gelling cationic chitosan solution that can be transformed into thermally sensitive, pH dependent, gel forming system by addition of polyol salts (β-glycerophosphate, GP) (this polysaccharide (sugar) system gel possess a neutral pH, remains liquid at or below normal average body temperature, and forms monolithic (continuous piece of gel) gels at body temperature).

In one embodiment of the present invention, the cooling composition of the present invention may further include a “phase change component” 24 or “PCC” 24 that may be initially suspended in the IPN 12. (While the IPN 12 is technically a phase change material, it will be differentiated from the PCC's 24 in the context of this invention.) A PCC 24 may be a mixture of two or more substances, such as two or more of the suitable phase change components.

A PCC 24 may melt at a specific Critical Temperature. That melting requires heat, which is taken from the skin, imparting a cooling to the skin of a mammal. Once the material is melted, the cooling dissipates to the point it is not detected by the mammal.

In one embodiment of the present invention, a particular PCC 24 having a Critical Temperature somewhere less than Core Body Temperature or Body Temperature, but above ambient Room Temperature, may be applied to a user's skin. The PCC 24 may cool the skin as it experiences a phase change. However, after the phase change has occurred, the cooling effect may no longer be detected by the user. Further, because a PCC 24 is commonly absorbed by the skin, the PCC 24 may not be removed. Once an absorbed PCC crosses the Critical Temperature, it will likely not be able to phase change again to create another cooling reaction.

In another embodiment of the present invention, a PCC 24 may be is combined with an IPN 12 in a way that the PCC could be recharged by the user. For instance, a PCC 24 having a Critical Temperature somewhere less than Core Body Temperature or Body Temperature, but above Room Temperature, can be combined with an IPN 12 that is liquid at Room Temperature. Once applied to a user's body, the IPN 12/PCC 24 can experience an increase in temperature. Once the PCC 24 reaches a Critical Temperature for that PCC, a phase chance occurs that may cool the user's skin. Simultaneously, the IPN 12 changes from liquid to solid at a corresponding Critical Temperature, which may or may not be about equal to the Critical Temperature of PCC 24. Once the IPN 12 is in a solid form, it may be peeled away from the user's skin and return to a liquid form. When this previously used combination is reapplied to the user's skin, the PCC 24 may once again cross the temperature threshold and cause a cooling effect detectable by the user.

In yet another embodiment, a selection of two or more different types of PCC 24 may be combined such that the effective cooling of the cooling compound 10 can be adjusted over a wider range for any desired application. A PCC 24 may include a copolymer made of two or more substances. In one example, a combination of phase change components having Critical Temperatures at 18° C., 28° C., and 35° C. would provide extended cooling to the skin as each phase change component in succession melts.

For the purposes of the present invention, PCC 24 materials that have a Critical Temperature between about 15 degrees C. and about 40 degrees C. are appropriate for use in cooling a mammal's skin, such as a human. In other embodiments of the present invention, materials may be chosen with Critical Temperatures between about 15 degrees C. and about 35 degrees C.; or between about 18 degrees C. and about 35 degrees C.; or between about 23 degrees C. and about 35 degrees C.; or between about 25 degrees C. and about 35 degrees C.; or between about 28 degrees C. and about 35 degrees C.; or within any other suitable range. Of course, these ranges may be most desirable with respect to human skin. The ranges may be adjusted for particular animals, such as dogs, cats, and other mammals or creatures.

PCC's 24 of the present invention may include phase change components in a variety of forms (e.g., molten form, dissolved in a solvent, and so forth). Suitable PCC's of the present invention include, by way of example and not by limitation, LURAPRET® phase change powder, a purified, encapsulated paraffin available from BASF Corporation of New Jersey, hydrocarbons (e.g., straight chain alkanes or paraffinic hydrocarbons, branched-chain alkanes, unsaturated hydrocarbons, halogenated hydrocarbons, and alicyclic hydrocarbons), waxes, oils, fatty acids, fatty acid esters, dibasic acids, dibasic esters, 1-halides, primary alcohols, aromatic compounds, anhydrides (e.g., stearic anhydride), ethylene carbonate, polyhydric alcohols (e.g., 2,2-dimethyl-1,3-propanediol, 2-hydroxymethyl-2-methyl-1,3-propanediol, ethylene glycol, polyethylene glycol, pentaerythritol, dipentaerythritol, pentaglycerine, tetramethylol ethane, neopentyl glycol, tetramethylol propane, monoaminopentaerythritol, diaminopentaerythritol, and tris(hydroxvmethyl)acetic acid), polymers (e.g., polyethylene, polyethylene glycol, polypropylene, polypropylene glycol, polytetramethylene glycol, and copolymers, such as polyacrylate or poly(meth)acrylate with alkyl hydrocarbon side chain or with polyethylene glycol side chain and copolymers comprising polyethylene, polyethylene glycol, polypropylene, polypropylene glycol, or polytetramethylene glycol), and mixtures thereof.

The percentage of PCC 24 components in the cooling composition may vary depending on the desired application of the cooling composition. As an example, the cooling composition may include about one percent by weight (1% w/w) to about one hundred percent by weight (100% w/w) PCC 24 components. In other aspects, the cooling compositions may include about one percent by weight (1% w/w) to about sixty percent by weight (60% w/w); about two percent by weight (2% w/w) to about fifty percent by weight (50% w/w); about five percent by weight (5% w/w) to about forty-five percent by weight (45% w/w); about ten percent by weight (10% w/wo about forty percent by weight (40% w/w); about twelve and one-half percent by weight (12.5% w/w) to about thirty-five percent by weight (35% w/w); and about fifteen percent by weight (15% w/w) to about thirty percent by weight (30% w/w); PCC 24 components. Therefore, while the ingredients in the cooling compositions may typically include about one percent by weight (1% w/w) to about sixty percent by weight (60% w/w) PCC 24 components, some variability in the types of PCC 24 components employed within the cooling compositions is acceptable so long as the cooling compositions provide sufficient immediate cooling of the skin, and possible reactivatable and/or long term cooling.

In another embodiment of the present invention, the cooling composition of the present invention may be made by adding a “neurosensory component” 22 or “NSC” 22 to IPN 12. The NSC 22 produces a perception of an immediate and/or long term cooling sensation on skin when the neurosensory component is applied to skin.

NSC's 22 for use herein include all neurosensory components for which the cooling sensation is a physiological or neurosensory effect due to the direct action of these agents on the nerve endings of the mammal body responsible for the detection of hot or cold, with no or a limited occurrence of actual temperature change on the surface of the mammal body. It is believed that these agents act as a direct stimulus on the cold receptors at the nerve endings, which in turn stimulate the central nervous system. In this way, a cooling sensation is simulated in the absence of real change in skin temperature. Due to the persistence of the stimulus, a long lasting cooling sensation is delivered even after removal of the cooling agent.

Suitable NSC's 22 of the present invention include icilin, menthol, menthol derivatives, an encapsulated cooling agent such as SALCOOL™ cooling composition from Salvona L.L.C., Dayton, N.J., menthyl lactate, menthyl salicylate, menthyl acetate, menthyl PCA, menthyl carbinol, methyl linalool, isoeugenol, methyl eugenol, ICE 1500™ cooling sensate available from Qarôma, Inc., Baytown, Tex., menthone glycerol ketal, menthoxypropane-1,2-diol, (-)-isopulegol, cubebol, N-substituted p-menthane carboxamides, icilin, mint, mint oils, cucumber, chamomile, aloe, comfrey, anise, sage, carboamides, ketals, carboxamides, cyclohexanol derivatives, and/or cyclohexyl derivatives. Additional suitable neurosensory components are described in “Cool without Menthol & Cooler than Menthol and Cooling Compounds as Insect Repellents” John C. Leffingwell, Ph.D., leffingwell.com/cooler_than_menthol.htm, Apr. 19, 2007, which is hereby incorporated herein by reference to the extent it is consistent (i.e., not in conflict) herewith.

The percentage of NSC's 22 in the cooling composition may vary depending on the desired application of the cooling composition. As an example, the cooling composition may include up to about 5% w/w neurosensory components. In other aspects, the cooling compositions may include about 0.5% w/w to about 5% w/w; about 1% w/w to about 4% w/w; about 1% w/w to about 3% w/w; or about 1% w/w to about 2% w/w neurosensory components. Therefore, while the ingredients in the cooling compositions may typically include up to about 5% w/w neurosensory components, some variability in the types of neurosensory components employed within the cooling compositions is acceptable so long as the cooling compositions provide the desired immediate and/or long term cooling of the skin.

The cooling composition 10 may further include an “evaporative cooling component” 26 or “ECC” 26, such as “unbound” water or alcohol. Unbound water is water that is not bound by a water-saturated IPN, e.g. a hydrogel. In some aspects, the water may be purified or distilled. Water provides relatively long-term cooling. Alcohol provides the cooling through the latent heat of evaporation from the alcohol. The alcohol may be isopropyl alcohol, ethyl alcohol, or any other suitable alcohol. Alcohol provides relatively short-term cooling.

Suitable ECC's of the present invention include water; hydrocarbons such as isododecane and isoeicosane; short chain alcohols such as ethanol and n-propanol; small branched chain alcohols such as isopropyl alcohol; fluorinated hydrocarbons such as perfluorodecalin, perfluoroheptane, perfluorohexane, and perfluoromethylcyclohexane; fluorinated alcohols such as C6-C12 perfluoroalkylethanol and perfluorocyclohexylmethanol; fluorinated ethers such as ethyl perfluorobutyl ether, ethyl perfluoroisobutyl ether, methyl perfluorobutyl ether, methyl perfluoroisobutyl ether, and perfluorohexylethyl dimethylbutyl ether; low molecular weight grades of dimethicone, particularly DOW CORNING® 200 dimethicone fluid 0.65 c st; volatile cyclomethicones such as octamethyl cyclotetrasiloxane, decamethyl cyclopentasiloxane, dodecamethyl cyclohexasiloxane, and tetradecamethyl cycloheptasiloxane.

The percentage ECC in the cooling composition may vary depending on the desired application of the cooling composition. As an example, the cooling composition may include up about 80% w/w alcohol. In other aspects, the cooling composition may include about 5% w/w to about 75% w/w, about 10% w/w to about 70% w/w, about 15% w/w to about 65% w/w, to about 20 to about 60% w/w, or about 25% w/w to about 50% w/w alcohol.

The cooling composition 10 may optionally include a surfactant that may promote emulsifying activity. Surfactants have the ability to lower the surface tension of water to reduce the interfacial tension between two immiscible substances. In some aspects, the surfactants in the cooling composition may enhance cleaning or removal of dirt, sweat, and/or sebum from the skin. Some surfactants may also act as a wetting agent to facilitate absorption of the cooling composition 10 on a substrate (e.g., clothes or tissue). In addition, some surfactants may act as emulsifying agents or solubilizing agents to emulsify or solubilize hydrophobic materials into hydro-alcohol formulations. Some variability in the types of surfactant employed within the cooling composition is acceptable so long as the surfactant provides sufficient emulsifying activity.

It may be noted that to achieve solubilization or emulsification of a lipophilic ingredient, the lipophilic ingredient (e.g., an oil soluble skin health benefit agent) will be compatible with a surfactant that is part of the cooling composition in order to obtain a stable formulation. As examples, surfactants may be selected from groups of sorbitan fatty acids (sorbitan monopalmitate, sorbitan monolaurate and the like), polyoxyethylene sorbitan fatty acid esters (polyoxyethylene 20 sorbitan monolaurate, polyoxyethylene sorbitan 20 monostearate, polyoxyethylene 4 sorbitan monostearate and the like), polyoxyethylene acids (polyoxyethylene 8 stearate, polyoxyethylene 20 stearate, and the like), and polyoxyethylene alcohols (polyoxyethylene 4 lauryl ether, polyoxyethylene 10 cetyl ether, polyoxyethylene 10 stearyl ether, polyoxyethylene 5.5 decyl ether, and the like), but the surfactant(s) can be selected from any suitable surfactants.

The cooling composition 10 may include other active or inactive additives depending on the desired application of the cooling composition. It may be noted that the absolute weight of any additive to the cooling composition may vary.

In some embodiments of the present invention, the cooling composition may include therapeutic additives. Some example therapeutic additives include anti-microbial agents, pain relievers, anti-inflammatory agents, skin-protectants, antiseptics, sunscreens, insect repellents, exfoliants, deodorants, antiperspirants, vitamins (e.g., vitamin B, C or E), and aloe vera (among others).

The cooling composition may also include an additive that regulates the release of one or more of the items which form the cooling composition at a desired rate. As an example, the additive may provide for long term delivery of one or more items in the cooling composition thus increasing the useful life of a product that includes the cooling composition. The appropriate amount of such an additive will depend on the desired rate and duration of the release. Examples of such additives include water insoluble polymers such as ethylcellulose, acrylic resins, co-polymer of methacrylic acid and acrylic acid ethyl ester, polylactic acid, polylactic-co-glycolic acid (PLGA), polyurethane, polyethylene vinyl acetate copolymer, and polystyrene-butadiene copolymer (or mixtures thereof).

Other additives may be included in the cooling composition to facilitate delivering the cooling composition to skin. Some examples of such additives include lubricants, plasticizing agents, preservatives, thickeners, emulsion stabilizers, stick formers, suppository formers, film formers, cream formers, coatings, binders, carrier, coloring agents, moisturizers, chelating agents, fragrance and/or odor controlling agents, humectants, viscosity controlling agents, and pH-adjusting agents (among others).

It may be noted that any number and type of additives may be included in the cooling composition. Some of the other example additives include potassium lactate, vitamin E, vitamin C, fragrance, botanicals, citric acid, sodium hydroxide, and/or potassium chloride (among others).

The cooling composition may be administered directly to the skin for prophylactic, therapeutic, and/or hygienic use. For example, the cooling composition may be administered in the form of a solution, liquid, bioadhesive gel, aerosol, foam, cream, gel, lotion, paste, jellies, or sprays.

When the cooling composition 10 is a liquid, the cooling composition may be administered from absorbent materials (e.g., a sponge, wipe, or pad). The cooling composition may also be administered as a spray/aerosol that is applied to the skin using a pump-type or aerosol sprayer. The cooling composition may also be administered using an applicator (e.g., a roll-on, squeeze-type, or plunger-type applicator).

In some aspects, the cooling composition may degrade slowly and remain attached to the skin for a period of time. It is contemplated that the cooling composition may even be absorbed into clothing or wiped off. It is further contemplated that the cooling composition may be peeled off. Such different methods of removal will largely depend on what phase the cooling composition is at the time of removal, either liquid or solid.

In various aspects of the present invention, the applications for the cooling compositions are many and varied. The examples as shown in FIGS. 1-3 are not meant to be limiting, and may include more than the types of additives, or more of the additives than is shown. Broadly speaking, the cooling compositions 10 can be classified as topical compositions, this term being taken in its broadest possible sense. Topical compositions include compositions such as perfumes and lotions applied to the external surfaces of the human body.

### EXAMPLE 1

Example 1 provides a cooling composition 10 that consists of an ICN 12. For example, an ICN in the form of a hydrogel may be made by combining two solutions. The first solution has the components of 2.2% w/w of medium viscosity chitosan and 2.2% w/w by of hydrochloric acid. The second solution has the components of 44% w/w water and 56% w/w of glycerol phosphate disodium salt hydrate (“GP”). The hydrogel is formed by bringing each solution to a temperature of about 0 C. (32 F.). This is not a necessary step, but may be done to slow the reaction and promote more uniform cross-linking of the gel, if desired. The GP solution is added drop wise to the chitosan solution while mixing. A flask containing the combined solution is cooled in ice water (0 C.) for about 20 minutes and mixed again at the end of this time period. The result is a transparent, thermally responsive, low odor or odorless hydrogel having a Critical Temperature (liquid to solid) of about 37 C. If desired, any air bubbles trapped in the hydrogel may be removed sonically as is known in the art.

The ICN or hydrogel may be applied to the skin by spreading with hand or a tool such as a roll-on applicator, a spatula, hand, towelette, spray, and any other known method of applying a relatively viscous liquid to the skin.

As the hydrogel is heated by the body and crosses a Critical Temperature (which in this particular example is about 37 C.), the hydrogel becomes a solid film. If the skin temperature falls below the Critical Temperature, the hydrogel returns to a liquid state. This may be repeated until the hydrogel is removed from the skin. Thus, the hydrogel may be peeled off of the skin when in a solid state, or wiped off from the skin in a liquid state.

### EXAMPLE 2

The ICN 12 of Example 1 may further include a NSC 24. In this example, menthol is included within the hydrogel matrix at about 0.5% w/w. As the hydrogel is applied, the menthol begins to trigger a cooling sensation by activating receptors within the skin. In this example cooling may occur from multiple sources as a result of the phase changes of the ICN 12, and the NSC 22. The resultant cooling effect is thereby enhanced for the consumer as multiple cooling mechanisms create a synergistic cooling effect and enhance the longevity of the cooling effect.

### EXAMPLE 3

The hydrogel of Examples 1 and 2 may further include an ECC 26 such as ethanol. In this example, the ethanol is included within the hydrogel matrix at about 25% w/w. As the cooling composition 10 is applied to skin such as human skin, the ethanol begins to flash and evaporate creating an immediate cooling sensation on the skin. The combination of multiple cooling agents within this embodiment will result in a cooling effect that is thereby enhanced for the consumer as multiple cooling mechanisms create a synergistic cooling effect and enhance the longevity of the cooling effect.

### EXAMPLE 4

The cooling composition 10 of Examples 1, 2, and 3 may further include a PCC 24 such as LURAPRET® 35 C. from BASF Corporation. In this example, the PCC 24 with a Critical Temperature of 35 C. is included within the ICN 12 at about 15% w/w. As the cooling composition 10 is applied to skin such as human skin, the PCC 24 cools the skin as a phase transition occurs.

These and other modifications and variations to the present invention may be practiced by those of ordinary skill in the art, without departing from the spirit and scope of the present invention, which is more particularly set forth in the appended claims. In addition, it may be understood that various aspects of the present invention may be interchanged either in whole or in part. Furthermore, those of ordinary skill in the art will appreciate that the foregoing description is by way of example only, and is not intended to limit the invention so further described in such appended claims.

What is claimed:

1. A temperature change composition for cooling the skin of a mammal, the composition comprising:
a thermo-responsive polymer comprising an interpenetrating polymer network that is in a liquid state at about Room Temperature, and a solid state at a temperature greater than about Core Body Temperature; and
at least one additional cooling component.

2. The composition of claim 1, wherein the interpenetrating polymer network comprises chitosan.

3. The composition of claim 1, wherein the interpenetrating polymer network comprises a hydrogel.

4. The composition of claim 1, wherein the at least one additional cooling component comprises a neurosensory component.

5. The composition of claim 4, wherein the neurosensory component is selected from the group consisting of menthol, menthol derivatives, menthyl lactate, menthyl salicylate, menthyl acetate, menthyl PCA, menthyl carbinol, methyl linalool, isoeugenol, methyl eugenol, menthone glycerol ketal, menthoxypropane-1,2-diol, (-)-isopulegol, cubebol, N-substituted p-menthane carboxamides, icilin, mint, mint oils, cucumber, chamomile, aloe, comfrey, anise, sage, carboamides, ketals, carboxamides, cyclohexanol derivatives and/or cyclohexyl derivatives.

6. The composition of claim 1, wherein the at least one additional cooling component comprises an evaporative cooling component.

7. The composition of claim 6, wherein the evaporative cooling component comprises an alcohol.

8. The composition of claim 6, wherein the evaporative cooling component is selected from the group consisting of a fluorinated hydrocarbon, a fluorinated ether, a low molecular weight grade of dimethicone, and a volatile cyclomethicone.

9. The composition of claim 1, wherein the at least one additional cooling component comprises a first phase change component.

10. The composition of claim 9 further comprising a second phase change component.

11. The composition of claim 9 further comprising a neurosensory component.

12. The composition of claim 1 having a Critical Temperature between about 15 degrees C. and 40 degrees C.

13. The composition of claim 1 further comprising a surfactant.

14. The composition of claim 1 further comprising therapeutic additives.

15. The composition of claim 1 wherein the at least one additional component comprises a neurosensory component, an evaporative component, and phase change component.

16. A temperature change composition for cooling the skin of a mammal comprising:
a thermo-responsive polymer comprising an interpenetrating polymer network that is in a solid state at Room Temperature; and
at least one additional cooling component.

17. A temperature change composition for cooling the skin of a mammal comprising:
a thermo-responsive polymer comprising a chitosan-based hydrogel; and
at least one additional cooling component selected from the group consisting of a phase change component, a first neurosensory cooling component, and an evaporative cooling component.

18. The composition of claim 17 further comprising a second phase change component.

19. The composition of claim 17 further comprising a second evaporative cooling component.

20. The composition of claim 17 further comprising a second neurosensory component.
